(19) United States
(12) Patent Application Publication
(10) Pub. No.: US 2011/0052608 A1
(43) Pub. Date: Mar. 3, 2011

(54) NOVEL ANTIBODIES AGAINST PRION PROTEIN AND USES THEREOF

(75) Inventors: Mark William Head, Edinburgh (GB); Michael Jones, Edinburgh (GB)

(73) Assignee: UNIVERSITY COURT OF THE UNIVERSITY OF EDINBURGH, Edinburgh (GB)

(21) Appl. No.: 12/304,525

(22) PCT Filed: Jun. 13, 2007
(86) PCT No.: PCT/GB2007/002205

§ 371 Date: Sep. 4, 2009

(30) Foreign Application Priority Data
Jun. 14, 2006 (GB) 0611708.9

(51) Int. Cl. A61K 39/395; C12N 15/06; C12N 5/12; G01N 33/566; G01N 33/567; C07K 16/44

(52) U.S. Cl. 424/175.1; 435/452; 435/345; 435/346; 436/501; 436/503; 530/388.9; 530/389.8

(57) ABSTRACT

The present invention relates to a novel antibody which selectively binds to the disease associated form of prion protein (PrP^{Sc}) under native conditions and the use thereof in methods of prion disease detection, therapy and disease research in general.

### FIELD OF THE INVENTION

The present invention relates to a novel antibody which selectively binds to the disease associated form of prion protein (PrP^{Sc}) under native conditions and the use thereof in methods of prion disease detection, therapy and disease research in general.

### BACKGROUND TO THE INVENTION

The prion diseases or transmissible spongiform encephalopathies (TSEs) are a group of rapidly progressive and fatal neurodegenerative disorders characterised by neuronal cell loss, spongiform change, gliosis and deposition of abnormal protein aggregates. Animal prion diseases include scrapie in sheep, bovine spongiform encephalopathy (BSE) in cattle and exotic ungulates, chronic wasting disease in deer and elk, transmissible mink encephalopathy and feline spongiform encephalopathy in domestic and exotic cats (Prusiner S. B. et al (1998) Cell 93:337-348). In humans, recognised prion diseases include kuru, sporadic Creutzfeldt-Jakob disease (sCJD), familial Creutzfeldt-Jakob disease (fCJD), Gerstmann-Straussler-Scheinker syndrome (GSS), fatal familial insomnia and variant CJD (vCJD) (Prusiner S. B. et al (1998) Cell 93:337-348). CJD has been transmitted between humans by contaminated cadaveric pituitary hormones, dura mater transplantation, neurosurgical instruments and corneal transplantation (Brown P. et al (2000) Neurology 55: 1075-1081). More recently evidence of vCJD transmission by blood transfusion has been reported (Llewelyn C. A. et al (2004) Lancet 363: 417-421; Peden A. H. et al (2004) Lancet 364: 527-529; Health Protection Agency (2006) CDR weekly 16 (6)) raising major concerns for both the transplantation and blood transfusion services. The need to develop highly sensitive and specific assays capable of detecting vCJD infectivity in tissues and blood has never been of greater importance. Such assays would not only be of value for disease diagnosis allowing early therapeutic intervention in the infected host prior to cell and tissue damage but also in the reduction/elimination of the risk of disease transmission by blood transfusion, blood products, surgery, dentistry and tissue/cell grafting/transplantation.

Prion diseases are characterised by conformational transitions of the normal cellular prion protein (PrP^{C}), which is found in most tissues, from its normal α-helical rich structure to abnormal β-sheet rich structures (PrP^{Sc}). Unlike PrP^{C}, PrP^{Sc} readily aggregates, is insoluble in non-ionic detergents and is partially resistant to limited proteinase K digestion resulting in the formation of a proteinase K resistant core (PrP^{res}) (Prusiner S. B et al (1998) Cell 93: 337-348). The presence of PrP^{Sc} is considered a hallmark of prion diseases, is closely associated with infectivity and is the only direct unambiguous disease-associated marker known to date (Morel N. et al (2004) J. Biol. Chem. 279: 30413-30149). Detection of PrP^{Sc} is therefore the basis of the majority of the diagnostic tests and screening assays currently being developed for human and animal applications. Given that test samples will contain both PrP^{C} and PrP^{Sc} reagents capable of distinguishing between the two forms under native conditions will be valuable tools in assay development. A number of such reagents have been described in the literature including plasminogen (Fischer M. et al (2000) Nature 408: 479-483), RNA aptamers (Rhie A. et al (2003) J. Biol. Chem. 278: 39697-39705; Sayer N. M. et al (2004) J. Biol. Chem. 278: 13102-13109)), anti-DNA antibodies and a DNA binding protein (Zou W. Q. et al (2004) PNAS 101: 1380-1385) and motif-grafted antibodies containing the replicative interface of PrP^{C} (Moroncini G. et al (2004) PNAS 101: 10404-10409). However, the most useful reagents in assay development will be antibodies which specifically bind to PrP^{Sc}.

Many of the anti-PrP antibodies currently available either cross-react with both PrP^{C} and PrP^{Sc} under native conditions, for example mAb 6H4 (Korth C. et al (1997) Nature 390: 74-77), or only bind to PrP^{C} under native conditions but bind to both PrP^{C} and PrP^{Sc} following denaturation in strong detergents, chaotropic reagents or by heating, for example mAb 3F4 (Kascsak R. J. et al (1987) J. Virol 61: 3688-3693). Indeed, these antibody binding properties have been utilised in the development of the so called conformational dependent immunoassay (CDI) (Safar J. et al (1998) Nat. Med. 4:1157-1165). However, in order to definitively distinguish between PrP^{C} and PrP^{Sc} it is necessary to pretreat test samples with proteinase K to completely degrade the PrP^{C} and allow detection of any PrP^{res} present in the sample. There is now mounting evidence that proteinase K sensitive forms of PrP^{Sc} also exist (Safar J. et al (2005) PNAS 102: 3501-3506) and that these forms are implicated in disease transmission (Yakovleva O. et al (2004) Transfusion 44: 1700-1705). Therefore antibodies capable of distinguishing between PrP^{C} and all forms of PrP^{Sc} without the need for proteinase K digestion are required.

Antibodies specifically recognise proteins via unique amino acid determinants or epitopes. These epitopes may be of a linear amino acid sequence or distinct conformations formed by amino acids in three-dimensional space. Considering conversion of PrP^{C} to PrP^{Sc} involves a major change in protein conformation it is likely that unique epitopes will be formed or revealed upon conversion. Attempts to produce antibodies specific for the native PrP^{Sc} by immunisation with purified PrP^{Sc} have generally been unsuccessful, with those antibodies characterised, for example 3F4 (Kascsak R. J. et al (1987) J. Virol. 61: 3688-3693), having little or no affinity for PrP^{Sc} under native conditions. However, several reportedly PrP^{Sc} specific antibodies have recently been described, mAb 15B3 raised against full-length recombinant bovine PrP (Korth C. et al (1997) Nature 390: 74-77), mAb V5B2 raised against a synthetic peptide corresponding to amino acid residues 214-226 of human PrP (Serbec V. C. et al (2004) J. Biol. Chem. 279: 3694-3698) and antibodies raised against a synthetic peptide comprising of the tyrosine-tyrosine-arginine motif found in PrP (Paramithiotis E. et al (2003) Nat. Med. 9: 893-899). However, the use of these antibodies outside of the laboratories in which they were produced is limited and the production of further PrP^{Sc} specific antibodies is merited.

There is mounting evidence that the region spanning amino acid residues 106-126 (numbering according to the human PrP sequence, Swiss-Prot primary accession number P04156) may be one of the key regions where conformational changes between PrP^{C} and PrP^{Sc} are initiated. Mice immunised with native PrP^{Sc} coated microbeads were shown to mount a predominantly IgM immune response targeting the region between PrP amino acid residues 101-120 suggesting that this region represented the major immunogenic region of native PrP^{Sc} (Tayebi M. et al (2004) Mol. Med. 10: 104-111). Furthermore, the antibody 3F4 (Kascsak R. J. et al (1987) J. Virol 61: 3688-3693), which binds to an epitope located between amino acid residues 109-112, can bind to native PrP^{C} but not to native PrP^{Sc} suggesting a major conformational change in this region upon conversion. Studies have shown that a synthetic peptide comprising amino acid residues 106-126 (PrP106-126) exhibited some of the properties associated with PrP^{Sc}. For example, PrP106-126 underwent a pH-dependent random coil to β-sheet transformation and aggregated to form amyloid fibrils that were partially resistant to digestion with proteinase K (Selvaggini F. et al (1993) Biochem. Biophys. Res. Commun. 194: 1380-1386, DeGioia L. et al (1994) J. Biol. Chem. 269: 7859-7862), and exposure of human neuronal cell lines to PrP106-126 micro-aggregates catalysed the aggregation of endogenous PrP^{C} to an amyloidogenic form that shared several characteristics with PrP^{Sc} (Singh N. et al (2002) Front. Biosci. 7: 60-71; Gu. Y. et al (2002) J. Biol. Chem. 277:2275-2286) and lead to cytotoxicity with features resembling prion toxicity. Without wishing to be bound by theory, the present inventors postulated that certain antibodies produced following immunisation of an animal with aggregated PrP106-126 might specifically bind to unique conformational epitopes formed upon aggregation and that these epitopes might also be present in PrP^{Sc}. This theory is supported in part by the publication of a paper describing the production of conformational-specific monoclonal antibodies (mAbs) that bound to the amyloid fibril state of the Alzheimer's disease peptide Aβ(1-40) but not to the soluble monomeric peptide (O'Nuallin B. et al (2002) PNAS 99: 1485-1490).

### SUMMARY OF THE INVENTION

The present invention is based in part on the use of aggregates, comprising of a synthetic peptide sequence corresponding to the human prion protein amino acid sequence from amino acid residues 106-126, to immunise animals, resulting in the production of certain antibodies capable of specifically detecting PrP^{Sc} but not PrP^{C} under native conditions without the need for prior proteinase K treatment of the sample.

Thus, in a first aspect there is provided use of an aggregated peptide comprising or consisting of the conserved amino acid sequence found between residues 106-126 of human PrP or the corresponding amino acid sequences from other species for raising antibodies specific thereto and in particular antibodies which are capable of binding to PrP^{Sc} and not PrP^{C}. In a further aspect, the invention provides an antibody, which is capable of selectively binding aggregated PrP106-126 and the abnormal disease associated PrP^{Sc} but not monomeric PrP106-126 and normal host PrP^{C}.

The antibody according to the present invention may be polyclonal or monoclonal and of the IgG. IgM, IgD, IgE, IgA isotype or fragments thereof.

Antibodies according of the present invention may also be humanised (Thompson, K. M. et al (1986) Immunology 58, 157-160) and/or of the single domain antibody form (Ward, E. S. et al (1989) Nature 341, 544-546).

Antibodies according to the present invention are capable of selectively binding to type 1 and type 2 PrP^{Sc} from sporadic CJD (sCJD) and vCJD in the presence of PrP^{C} without the need for prior proteinase K digestion to distinguish between PrP^{Sc} and PrP^{C}. Given the mounting evidence for the existence of proteinase K sensitive forms of PrP^{Sc} and the possible involvement of these forms in disease transmission a reagent capable of distinguishing between PrP^{Sc} and PrP^{C} without the need for prior proteinase K digestion is highly desirable.

Typically the antibody according to the present invention has been raised against a conformational epitope formed upon the aggregation of a PrP peptide fragment which is not found in the monomeric peptide. Preferably the peptide comprises or consists of the conserved amino acid sequence found between residues 106-126 of human PrP (Swiss-Prot primary accession number P04156) or the corresponding amino acid sequences from other species. For example, the corresponding sequence in mice lies between amino acid residues 105-126 (Swiss-Prot primary accession number P04925), in sheep between residues 109-129 (Swiss-Prot primary accession number P23907) and in cattle between residues 117-137 (Swiss-Prot primary accession number P10279). The skilled addressee can easily identify the corresponding sequences from other species. Peptides according to the invention may be synthesised by standard peptide synthesis techniques, for example using either standard 9-fluorenyl-methoxycarbonyl (F-Moc) chemistry, standard butyloxycarbonate (T-Boc) chemistry or the fluorenylmethoxycarbonyl (Fmoc)/tert-butyl system (Atherton E. and Sheppard R. C. (1998) Solid Phase Peptide Synthesis: A Practical Approach, Oxford, IRL Press). Purity, which will normally be in excess of 85%, should be carefully checked and various chromatographic techniques, including high performance liquid chromatography, and spectrographic analyses, including Raman spectroscopy, may for example be employed for this purpose. The peptide may be resuspended in a suitable buffer, for example PBS pH7.0, and incubated at room temperature for 16 hours to allow formation of aggregates prior to immunisation of suitable animals.

The present inventors have produced a hybridoma cell line which is capable of producing a monoclonal antibody according to the present invention. A synthetic peptide corresponding to the amino acid sequence 106-126 of human PrP (PrP106-126) was allowed to aggregate. This aggregated peptide was used to immunise mice. Splenocytes from said immunised mice were fused to a suitable mouse myeloma partner and hybridoma cell lines selected according to well-known techniques (Hawlow E. and Lane D. (1988) Antibodies: A Laboratory Manual, Cold Spring Harbour Lab. Press, Plainview, N.Y.). Supernatant samples from these hybridoma cell lines were screened for binding to aggregated PrP106-126 and monomeric PrP106-126 by ELISA. Those hybridoma cell lines secreting antibody which bound to aggregated PrP106-126 but not to monomeric PrP106-126 were then single cell cloned and frozen stocks of the cell line laid down.

Thus, in a third aspect of the present invention there is provided a hybridoma cell line (P1:1) capable of secreting a monoclonal antibody (IgM isotype) according to the present invention. This hybridoma cell line has been deposited with the ECACC, in accordance with the Budapest Treaty, on the 6 Jun. 2006 and is available under the accession number 06060601.

Antibodies according to the present invention are, inter alia, of use in a method of detecting the presence of disease-associated PrP^{Sc} in tissues (for example brain, tonsil or spleen tissue biopsy extracts) and bodily fluids (for example blood, CSF) in the presence of PrP^{C} without the need for prior proteinase K digestion. Accordingly, there is provided a method of detecting PrP^{SC} in a sample, comprising the steps of:
a) providing a sample of tissue or bodily fluid;
b) contacting said sample with an antibody according to the present invention so that the antibody is able to bind any PrP^{Sc} present in the sample; and
c) detecting whether or not PrP^{Sc} is present in the sample by virtue of detecting antibody-PrP^{Sc} immune-complexes.

In respect to the detection of antibody-PrP^{Sc} immune-complexes the skilled person will be aware of a variety of immunoassay techniques known in the art, inter alia, ELISA, DELFIA, RIA, immunoprecipitation followed by Western blotting and flow-cytometry. Any appropriate label including, without limitation any radioactive, fluorescent, chromogenic (for example alkaline phosphatase or horseradish peroxidase), chemiluminescent or a hapten (for example biotin) which may be directly or indirectly visualised may be used in these immunoassays. The antibody according to the present invention may be labelled directly for use in immunoassays. Alternatively, the complex formed by PrP^{Sc} and the antibody according to the present invention may be detected indirectly either using a suitably labelled anti-mouse immunoglobulin or a suitably labelled anti-PrP antibody

Thus, a fourth aspect the present invention provides a method or kit for the detection of disease associated PrP^{Sc} in animal, especially human tissue and bodily fluids which comprises at least in part an antibody of the present invention.

Given the relative conservation of the peptide sequence in different species it is predicted that antibodies of the present invention would cross-react with PrP^{Sc} from other species. To test this theory we were able to show that an antibody from the present invention could specifically bind to aggregated recombinant mouse PrP but not monomeric recombinant mouse PrP by immunoblotting.

Thus, a fifth aspect to the present invention provides a method of detecting PrP^{Sc} from other species using antibodies according to the present invention and includes any methods/kits comprising at least an antibody of the present invention.

A sixth aspect to the present invention includes the use of the antibody according to the present invention for determining the efficacy of putative prion disease therapeutic agents. The antibody of the present invention can be used, inter alia, in assays for the detection of PrP^{Sc} to determine if potential therapeutic agents can clear infectivity or prevent its occurrence in both cell culture and animal models.

A number of papers have recently reported that certain anti-PrP antibodies can inhibit prion replication and delay the development of prion disease both in vivo (White A. R. et al (2003) Nature 422: 80-83; Sigurdsson E. M. et al (2003) Neuroscience Letters 336: 185-187) and in vitro (Feraudet C. et al (2005) J. Biol. Chem. 280: 11247-11258). Thus, in a seventh aspect to the present invention provides the use of any form of the antibody according to the present invention for the manufacture of a medicament the treatment or prophylaxis of prion diseases in humans and/or other species.

### DETAILED DESCRIPTION

The present invention will now be described in detail by way of example and with reference to figures. These examples serve to illustrate particular embodiments of the present invention and they should not be considered a limitation thereof.

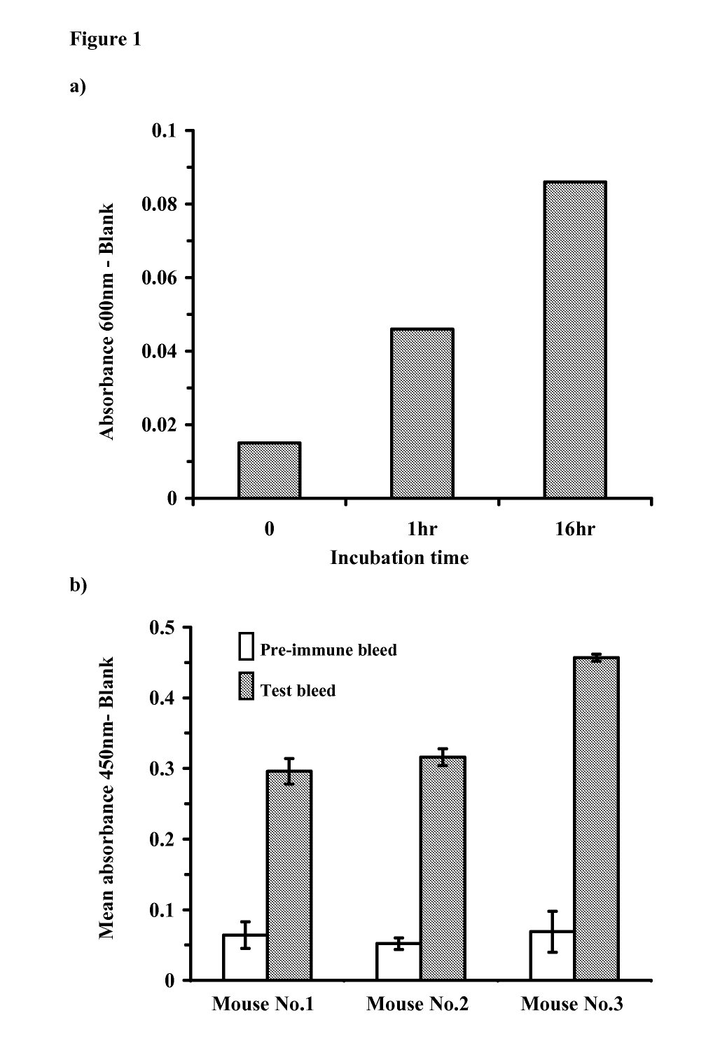

FIG. 1. Production of PrP106-126 aggregates and immunisation of PrP null mice

a) Time course of formation of PrP106-126 aggregates as monitored by increasing turbidity at 600 nm for samples taken at time 0, after 1 hour incubation and after 16 hours incubation at room temperature.

b) Immune response of each mouse immunised with aggregated PrP106-126 as determined by serum antibody binding to aggregated PrP106-126 coated microwells by ELISA for pre-immune serum samples (white bars) and test bleed serum samples (grey bars). The increase in absorbance values obtained for test-bleed serum samples compared to the pre-immune serum samples was indicative of a positive immune response.

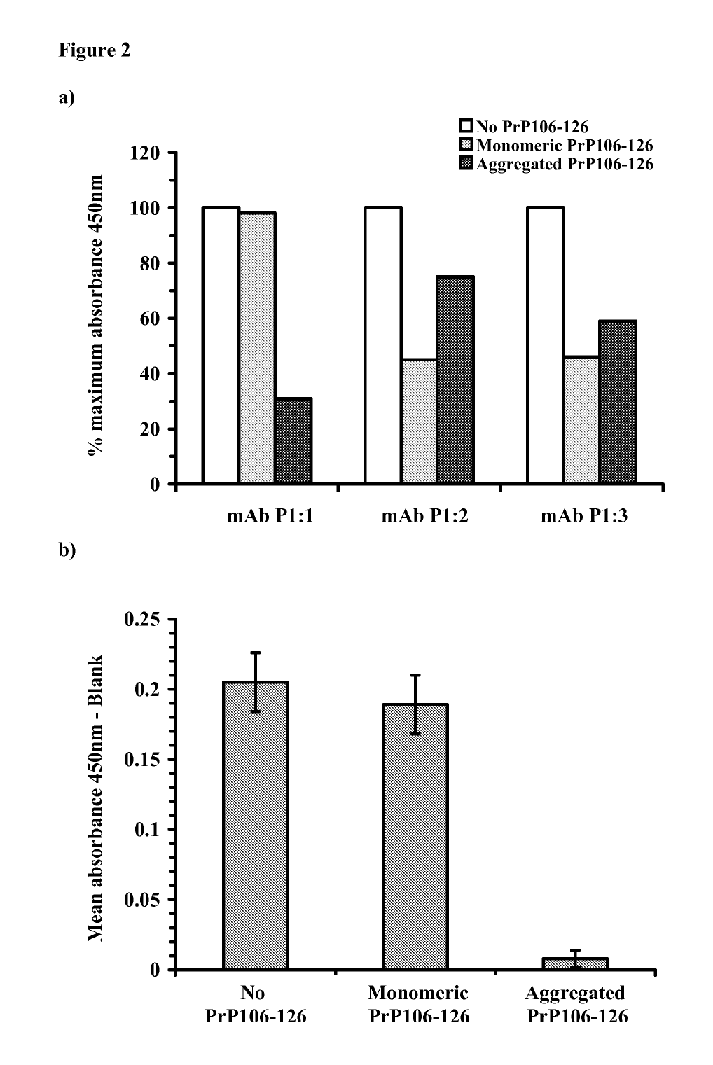

FIG. 2. Identification of mAbs specifically binding to aggregated PrP106-126

a) mAbs P1:1, P1:2 and P1:3 were pre-incubated in the absence of PrP106-126 peptide (white bars), in the presence of monomeric PrP106-126-NH₂ (grey bars), in the presence of aggregated PrP106-126 (black bars) and then screened for antibody binding to aggregated PrP106-126 coated microwells by ELISA. Results obtained were expressed as % maximum absorbance in order to normalise the results for all three mAbs. Binding of mAbs P1:2 and P1:3 were inhibited following pre-incubation with both monomeric and aggregated PrP106-126. Whereas, binding of mAb P1:1 was only inhibited following pre-incubation with aggregated PrP106-126.

b) ELISA showing the specificity of purified mAb P1:1 for binding to aggregated PrP106-126. mAb P1:1 was pre-incubated in the absence of PrP106-126 peptide, in a 100-fold molar excess of monomeric PrP106-126-NH₂, in a 100-fold molar excess of aggregated PrP106-126 and then screened for binding to aggregated PrP106-126 coated microwells Inhibition of binding was only detected following pre-incubation with a 100-fold molar excess of aggregated PrP106-126.

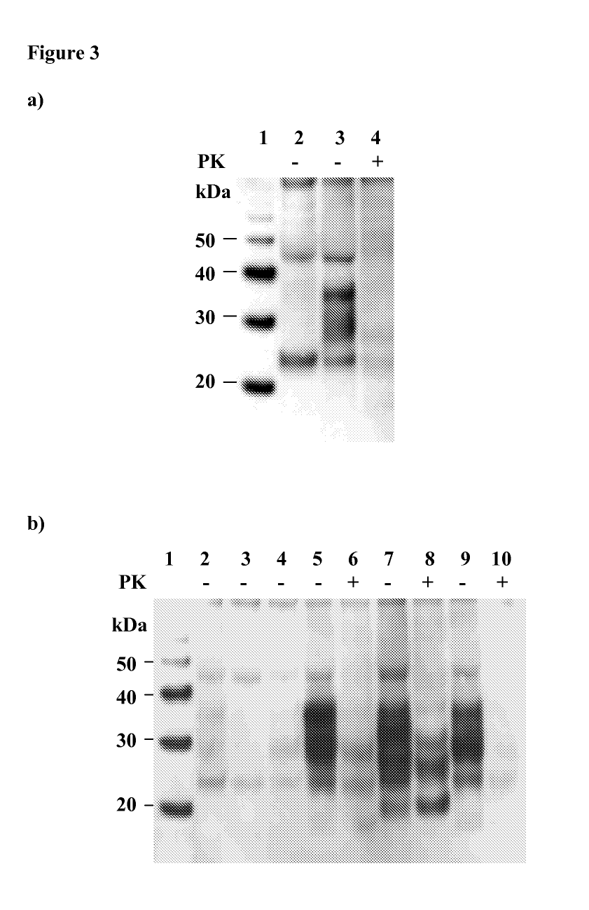

FIG. 3. Immunoprecipitation of PrP^{C}, PrP^{Sc} and PrP^{res} from human brain homogenate

a) 1% brain homogenates from an Alzheimer's disease neurological control brain (Lane 2) and a vCJD brain (Lanes 3 and 4) were immunoprecipitated with 10 μg mAb P1:1. For Lane 4 the vCJD brain homogenate was treated with proteinase K (PK) prior to immunoprecipitation. Following SDS-PAGE alongside molecular weight markers (Lane 1) and electrotransfer onto PVDF membrane PrP^{C}, PrP^{Sc} and PrP^{res} were detected by probing the membrane with mAb 3F4.

b) 1% homogenates prepared from Lewy body dementia (Lane 2), Alzheimer's disease (Lane 3), amyloid angiopathy (Lane 4), vCJD (Lanes 5 and 6), sCJD MM1 (Lanes 7 and 8) and sCJD VV2A (Lanes 9 and 10) brains were immunoprecipitated with mAb P1:1. For Lanes 6, 8 and 10 brain homogenates were digested with PK prior to immunoprecipitation. Following SDS-PAGE alongside molecular weight markers (Lane 1) and electrotransfer onto PVDF membrane PrP^{C}, PrP^{Sc} and PrP^{res} were detected by probing the membrane with mAb 3F4.

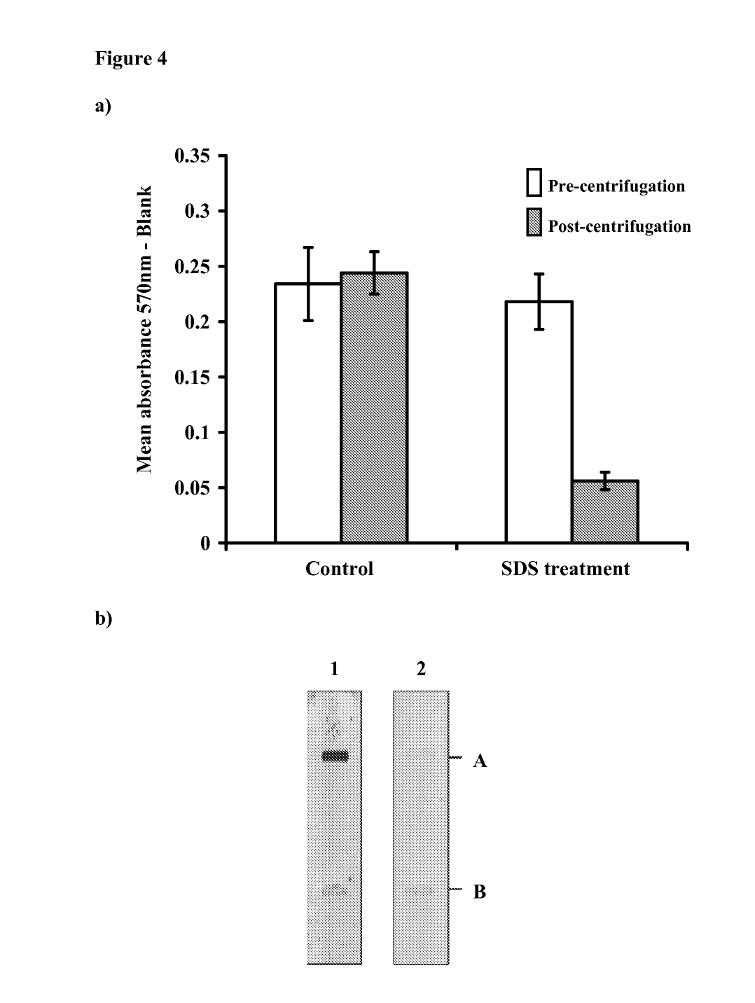

FIG. 4. Production of recMoPrP aggregates and comparison of the binding of mAb P1:1 to monomeric and aggregated recMoPrP

a) α-helical recMoPrP was prepared in either PBS (Control) or PBS+0.2% SDS (SDS-treated) and incubated for 10 min at room temperature. Both samples were then diluted 20-fold in PBS and incubated overnight at room temperature. Aliquots of each sample were spun down at 14,000×g for 30 min and the supernatants collected. The protein content in each sample pre-centrifugation (white bars) and supernatant post-centrifugation (grey bars) was determined by BCA protein assay with the results expressed as the mean absorbance 570 nm reading obtained. Decrease in the absorbance 570 nm reading obtained for the SDS-treated sample post-centrifugation compared to the reading pre-centrifugation was indicative of the formation of insoluble recMoPrP aggregates,

b) Aliquots of aggregated (Slot A) and monomeric (Slot B) recombinant mouse PrP, as described above, were slot blotted onto nitrocellulose membrane and probed with either mAb P1:1 (Blot 1) or a non-PrP related mouse IgM as a negative control (Blot 2). mAb P1:1 specifically bound to aggregated recMoPrP.

### EXAMPLE 1

### Preparation of PrP106-126 Amyloid Fibrils

Synthetic peptides corresponding to human PrP106-126 (KTNMKHMAGAAAAGAVVGGLG) and PrP106-126-NH₂ (KTNMKHMAGAAAAGAVVGGLG-NH₂) were obtained from Sigma Genosys. To produce aggregated PrP106-126, PrP106-126 (2 mg) was added to 1 ml 200 mM phosphate buffer, pH 7.0 and incubated at room temperature for 16 hours. Aggregate formation was monitored by taking aliquots (125 μl), at time 0, 1 hour and 16 hours, diluting to a 1 ml final volume in phosphate buffer and measuring the turbidity at 600 nm against a phosphate buffer blank. Aggregated PrP106-126 was formed as described, monitoring aggregate formation by measuring the change in turbidity at 600 nm (FIG. 1a), and used to immunise PrP null mice.

### EXAMPLE 2

### Immunisation of Mice

Three PrP null (PrP^{-/-}) mice (supplied by the Neuropathogenesis Unit, IAH, Edinburgh) (Manson J. C. et al (1994) Mol. Neurobiol. 8: 121-127) were each immunised subcutaneously with 50 μg aggregated PrP106-126 in Complete Freunds adjuvant followed by two further booster subcutaneous immunisations of 50 μg aggregated PrP106-126 in Incomplete Freunds adjuvant at 28-day intervals. Seven days after the final immunisation test bleeds were taken and the resulting serum samples screened for antibody binding to aggregated PrP106-126 coated microwells by ELISA as described below.

Wells of a 96-well Immulon 4 HXB microtitre plate (Thermo Labsystems) were coated with 100 ng aggregated PrP106-126 in 10 mM phosphate, 2.7 mM KCl, 137 mM NaCl, pH 7.4 (PBS) overnight at 37° C. The wells were washed three times with phosphate buffered saline containing 0.05% (v/v) Tween 20 (PBST), blot dried, blocked with 5% fetal calf serum (FCS) in PBST (200 μl/well) for 60 minutes at 37° C., the wells washed twice with PBST and blot dried. Aliquots (100 μl/well) of test serum and pre-immune serum from the three immunised mice, at a 1/1000 dilution in PBST+1% FCS, were added to triplicate wells, incubated at 37° C. for 60 minutes, the wells washed four times with PBST and blot dried. Goat anti-mouse polyvalent Ig HRP conjugate (Sigma), at a 1/2000 dilution in PBST+1% FCS (100 μl/well) was added to all wells, incubated at 37° C. for 60 minutes, the wells washed four times with PBST and blot dried. SureBlue TMB Microwell Peroxidase Substrate (Insight Biotechnology Ltd) (100 μl/well) was added to all wells and incubated at 37° C. for 30 minutes at which point 0.18M sulphuric acid stop solution (100 μl/well) was added and the absorbance at 450 nm measured using a microplate reader (Dynex MRX). Results were calculated as the mean absorbance 450 nm for each test sample corrected for the mean absorbance for non-specific binding of the anti-mouse polyvalent Ig HRP to PrP106-126 fibril coated wells.

All three immunised mice mounted an immune response against aggregated PrP106-126 as determined by ELISA screening of pre-immune and final test bleed serum samples from each mouse for antibody binding to aggregated PrP106-126 coated microwells (FIG. 1b).

### EXAMPLE 3

### Production of Monoclonal Antibodies

The mouse chosen for hybridoma production received a final intravenous boost of 50 μg aggregated PrP106-126 in PBS and was sacrificed four days later. Splenocytes from the immunised mouse were fused with SP2/0-Ag14 mouse myeloma cells (ECACC No. 85072401) using a conventional polyethylene glycol (PEG) 1500 fusion protocol and the resulting hybridomas were selected in HAT medium (Hawlow E. and Lane D. (1988) Antibodies: A Laboratory Manual, Cold Spring Harbour Lab. Press, Plainview, N.Y.). Hybridoma supernatants were routinely screened for the secretion of mAbs binding to aggregated PrP106-126 by ELISA essentially as described to determine the immune response, except that supernatant samples were screened at ½ dilution in PBST+1% FCS. Hybridomas secreting mAbs binding to aggregated PrP106-126 were single cell cloned three times and frozen stocks of each cell line laid down. The isotypes of the mAbs produced were determined using the Isostrip mouse monoclonal antibody isotyping kit (Roche Diagnostics) as per the instructions supplied with the kit.

Out of the 360 wells seeded, post fusion of splenocytes from Mouse No. 3 to SP2/0 mouse myeloma cells, 4 wells were identified as containing actively dividing hybridomas secreting antibodies which bound to aggregated PrP106-126 coated microwells (results not shown). Cells from these 4 wells were single cell cloned and named P1:1, P1:2, P1:3 and P1:4 respectively. It was noted during the cloning that cell line P1:4 grew extremely slowly and no further work with this cell line was carried out once frozen stocks of the cell line were laid down.

Hybridomas P1:2 and P1:3 were both shown to secrete IgG1, kappa isotype antibodies. Hybridoma P1:1 secreted an IgM, kappa isotype antibody, although a very weak IgG1 signal was also detected with this cell line that could not be eliminated even after further rounds of single cell cloning and we now suspect that this was caused by non-specific interaction with the Roche isotyping strips.

### EXAMPLE 4

### Identification of mAbs Specifically Binding to Aggregated PrP106-126

Aliquots of each hybridoma supernatant were mixed 1:1 with PBST+1% FCS, aggregated PrP106-126 (10 μg/ml final concentration) in PBST+1% FCS or monomeric PrP106-126-NH₂ (10 μg/ml final concentration) in PBST+1% FCS, incubated on a roller mixer at room temperature for 60 minutes and spun down at 14,000×g for 10 minutes. COOH-terminal amidation had been reported to decrease the propensity of PrP106-126 to form aggregates (Salmona M. et al (1999) Biochem. J. 342:207-214; Bergstrom A. L. et al (2005) J. Biol. Chem. 280: 23114-23121)) and it was assumed that the peptide PrP106-126-NH₂ would therefore not form aggregates especially when freshly prepared just prior to use. Aliquots (100 μl) of each supernatant were transferred to triplicate wells of a aggregated PrP106-126 coated microtitre plate and the ELISA carried out as previously described. Hybridomas showing a significant decrease in antibody binding following preincubation with aggregated PrP106-126 but not with monomeric PrP106-126-NH₂ were taken forward for further analysis.

From the results obtained (FIG. 2a), it was evident that preincubation with both aggregated PrP106-126 and the PrP106-126-NH₂ monomeric peptide inhibited the binding of mAbs P1:2 and P1:3 to aggregated PrP106-126 coated microwells. This suggested that mAbs P1:2 and P1:3 bound to epitopes present in both the aggregated and monomeric PrP106-126. It would appear that these two mAbs have similar properties to a mAb previously produced against PrP106-126 following immunisation of mice with the peptide conjugated to keyhole limpet hemocyanin (Hanan E. et al (2001) Cell Mol. Neurobiol. 21: 693-703).

Binding of mAb P1:1 to aggregated PrP106-126 coated microwells was only inhibited following preincubation with aggregated PrP106-126 and not with the monomeric peptide (FIG. 2a). mAb P1:1 therefore appeared to bind to a conformational epitope specific for aggregated PrP106-126. It is interesting to note that the isotype of mAb P1:1 (IgM, kappa) was the same as the two mAbs previously reported to specifically bind to Alzheimer's peptide Aβ(1-40) fibrils (O'Nuallain B. et al (2002) PNAS 99:1485-1490).

Based on these results it was decided to stop all work on hybridomas P1:2 and P1:3 once frozen stocks of each cell line had been laid down and to concentrate on hybridoma P1:1. The specific binding of the purified mAb P1:1 to aggregated PrP106-126, at a concentration of 1 μg/ml mAb P1:1, was confirmed by ELISA (FIG. 2b). Of some concern were the relatively low absorbance values obtained, maximum absorbance reading of 0.205 in the absence of inhibitor. These low readings could be due to the fact that the mAb P1:1 only has a low affinity for its target, which is not uncommon for IgM isotype antibodies. However, the possibilities of low availability of specific epitopes or steric hindrance should not be discounted and requires further investigation.

### EXAMPLE 5

### Purification of IgM Isotype Monoclonal Antibodies

IgM isotype mAbs were purified from spent hybridoma culture supernatant (200 ml) by precipitation with 50% saturated ammonium sulphate followed by size exclusion chromatography on a Superose 6 column (Amersham Biosciences) into PBS buffer. The purified IgM concentration was then determined by ELISA as follows.

Wells of a 96-well Immulon 4 HXB microtitre plate were coated with 100 ng/well anti-mouse (μ-chain specific) IgM (Sigma) in 50 mM carbonate/bicarbonate coating buffer, pH 9.6 overnight at 4° C. The wells were washed three times with PBST, blot dried, blocked with 200 μl/well PBST+5% FCS at 37° C. for 60 minutes, washed twice with PBST and blot dried. Two-fold serial dilutions of a mouse IgM standard (Sigma) were prepared in the range 200 ng/ml to 1.56 ng/ml and two-fold serial dilutions of the test sample were prepared at a 1/1000 to 16,000 dilution, with all dilutions prepared in PBST+1% FCS. Aliquots (100 μl of each standard and test sample were transferred to wells of the anti-mouse IgM coated microtitre plate in triplicate, incubated at 37° C. for 60 minutes, the wells washed four times with PBST and blot dried. Goat anti-mouse (μ-chain specific) IgM HRP conjugate (100 μl/well) at a 1/2000 dilution in PBST+1% FCS was added to all wells, incubated at 37° C. for 30 minutes, the wells washed four times with PBS and blot dried. TMB substrate (100 μl/well) was added to all wells and incubated at room temperature for 10 minutes at which point acid stop solution (100 μl/well) was added and the absorbance at 450 nm measured using a microplate reader. The mean absorbance of each standard and test sample was calculated and the mean absorbance for each standard plotted against the corresponding IgM concentration to produce a standard curve from which the IgM concentration in the test sample was determined.

mAb P1:1 was purified as described resulting in the recovery of 1.36 mg IgM from 200 ml spent hybridoma medium. The final concentration of the purified IgM was adjusted to 1 mg/ml with PBS, maltose 10% (w/v) and sodium azide 0.001% (w/v) were added and the purified IgM stored in 100 μl aliquots at −40° C.

### EXAMPLE 6

### Immunoprecipitation of PrP^{C}, PrP^{Sc} and PrP^{res} from Human Brain Homogenate

Brain homogenates (10%) from an Alzheimer's Disease neurological control brain and a vCJD brain were prepared in 0.5% NP-50, 0.5% sodium deoxycholate, Tris buffered saline (TBS), pH7.4. The homogenates were centrifuged at 200 rpm for 5 minutes and the supernatants collected. For proteinase K digestion (PK), PK was added to clarified homogenate at a final concentration of 50 μg/ml, incubated at 37° C. for 60 minutes and the digestion stopped by the addition of Pefabloc (1 mM final concentration). Aliquots (10 μl) of 10% brain homogenates from the Alzheimer's Disease neurological brain homogenate (non-PK treated) and the vCJD brain homogenate (both non-PK and PK treated) were mixed with 10 μg mAb P1:1 in 100 μl final volumes in 10 mM phosphate, 2.7 mM KCl, 137 mM NaCl, pH 7.4 (PBS) and incubated on a rotary mixer overnight at 4° C. Rat anti-IgM conjugated Dynabeads (10 μl) (Dynal) were added to each sample and mixed on a rotary mixer for 60 min at room temperature. The beads were then washed three times in PBS, resuspended in 25 μl 1×NuPAGE LDS sample buffer (Invitrogen) and boiled for 10 min.

The immunoprecipitates were loaded on a NuPAGE Novex 10% Bis-Tris gel (Invitrogen), subjected to electrophoresis at 200V constant voltage for 45 min and electrotransferred onto PVDF membrane at 30V constant voltage for 60 min. The membrane was blocked in 5% dried milk powder in 10 mM Tris-HCl, 150 mM NaCl, 0.05% (v/v) Tween 20, pH 7.5 (TBST) overnight at 4° C. Following two washes in TBST (3 minutes per wash) the membrane was incubated in mAb 3F4 (Dako), at a 1/1000 dilution in TBST, for 60 min at room temperature then washed three times in TBST (3 minutes per wash). The membrane was then incubated in goat anti-mouse IgG (Fab-specific) peroxidase conjugate (Sigma), at a 1/40,000 dilution in TBST, for 60 min at room temperature. Following three washes in TBST (3 minutes per wash) the membrane was incubated in ECL Plus reagent (Amersham Biosciences) for 5 min at room temperature, the membrane drained, placed between two sheets of transparency film and exposed to Hyperfilm ECL (Amersham Biosciences) for 30 sec, 3 min and 10 min exposures. The Hyperfilm was then developed using a Hyperprocessor. All subsequent immunoprecipitation experiments using other non-PK treated neurological control (amyloid angiopathy and Lewy body dementia), sCJD MM1 (both non-PK and PK treated) and sCJD VV2 (both non-PK and PK treated) brain homogenates were carried out as described above.

Initial experiments (FIG. 3a) showed that mAb P1:1 immunoprecipitated PrP^{Sc} from the non-PK treated vCJD brain homogenate (FIG. 3a, Lane 3) and trace amounts of PrP^{res} from the PK treated vCJD homogenate (FIG. 3a, Lane 4). No PrP^{C} was immunoprecipitated from the Alzheimer's Disease brain homogenate (FIG. 3a, Lane 2). Based on this data it appeared that mAb P1:1 selectively immunoprecipitated full-length PrP^{Sc} from the vCJD brain but not PrP^{res} following PK digestion. This observation was similar to that made for mAb 15B3 (Korth C. et al (1997) Nature 390:74-77), a mAb raised against full-length recombinant bovine PrP, which is reported to specifically bind to PrP^{Sc}. mAb 15B3 appeared to more efficiently immunoprecipitate intact PrP^{Sc} compared to PK digested PrP^{res} and it was suggested that this was due to the fact that PK digestion resulted in the formation of large aggregates (scrapie-associated fibrils) which might mask the 15B3 epitope. Whilst this explanation might also be true for mAb P1:1, other possible explanations needed to be considered. One possibility was that PK digestion prior to immunoprecipitation could alter the conformation of PrP^{res} compared to PrP^{Sc} thus disrupting the conformational epitope recognised by mAb P1:1. Another more intriguing possibility was that mAb P1:1 might specifically immunoprecipitate a PK sensitive form of PrP^{Sc} present in the vCJD brain homogenate.

To further investigate these possibilities we attempted to immunoprecipitate PrP^{Sc} and PrP^{res} not only from a vCJD brain homogenate but also from sCJD MM1 and sCJD VV2 brain homogenates. In addition, in order to confirm the specificity of mAb P1:1 for PrP^{Sc}, we included two additional neurological control brain homogenates (amyloid angiopathy and Lewy body dementia) alongside the Alzheimer's disease brain homogenate. From the results obtained (FIG. 3b) in was evident that mAb P1:1 failed to immunoprecipitate PrP^{C} from any of the neurological control brain homogenates (FIG. 3b, Lanes 2, 3 and 4). mAb P1:1 immunoprecipitated full-length PrP^{Sc} from the vCJD brain (FIG. 3b, Lane 5), sCJD MM1 brain (FIG. 3b, Lane 7) and sCJD VV2 brain (FIG. 3b, Lane 9) homogenates in the absence of PK digestion. Following PK digestion, mAb P1:1 only immunoprecipitated trace amounts of PrP^{res} from the vCJD brain (FIG. 3b, Lane 6) and the sCJD VV2 brain homogenates (FIG. 3b, Lane 10), however, significantly more PrP^{res} was immunoprecipitated from the sCJD MM1 brain homogenate (FIG. 3b, Lane 8). Thus it would appear that mAb P1:1 preferentially bound to both Type 1 and Type 2 full-length PrP^{Sc} and Type 1 PrP^{res} but very weakly to Type 2 PrP^{res}. These observations appeared to suggest that the binding of mAb P1:1 to PrP^{Sc} and PrP^{res} was influenced by the PrP NH₂-terminal region (amino acids 23-97). One of the main differences between Type 1 and Type 2 PrP^{Sc} is the location of the primary PK cleavage site, located at residue 82 for Type 1 PrP^{Sc} and at residue 97 for Type 2 PrP^{Sc} (Parchi P. et al (2000) PNAS 97: 10168-10172). Thus, following PK digestion Type 1 PrP^{res} would have a slightly longer NH₂-terminal than Type 2 PrP^{res}. Based on the results obtained with mAb P1:1 it would appear that PK digestion resulted in a change in conformation of the resulting PrP^{res} compared to full-length PrP^{Sc}, indeed such a change in conformation had previously been reported (Safar J. et al (1993) J. Biol. Chem. 268: 20276-20284) due to an apparent reshuffling of the residual protein structure, and that this change in conformation was influenced by the primary site of PK cleavage.

### EXAMPLE 7

### Antibody Binding to Recombinant Mouse PrP Fibrils

For recombinant PrP solubilised in 0.2% SDS it had been reported that the recombinant PrP underwent a conformational change forming large multimers exhibiting proteinase K resistance upon reduction of the SDS content to less than 0.01% and prolonged incubation at room temperature (Post K. et al (1998) Biol. Chem. 379: 1307-1317). We therefore adapted a previously described method (Trieschmann L. et al (2005) BMC Biotechnol. 5: 26-30) to produce recombinant mouse PrP (recMoPrP) aggregates from monomeric α-helical recMoPrP. α-helical recombinant mouse PrP (supplied by Dr Andy Gill, IAH, Compton) was prepared in both PBS and PBS+0.2% (w/v) SDS to give a final recMoPrP concentration of 100 μg/ml and incubated at room temperature for 10 minutes. Both samples, with and without SDS, were diluted twenty-fold in PBS and incubated overnight at room temperature. Formation of recMoPrP aggregates was determined as follows: aliquots (1 ml) of each sample were spun down at 14,000×g for 30 minutes and the resulting supernatants collected. The protein distribution in the samples pre-centrifugation and the supernatants post centrifugation were then determined using the reagents supplied in the Pierce BCA Protein Assay Kit. Briefly, 25 μl of each sample pre-centrifugation and supernatant samples post-centrifugation were added to 200 μl BCA reagent, incubated at 37° C. for 30 minutes and the absorbance at 570 nm measured. The fact that the recMoPrP could be spun down out of solution following SDS treatment but not following incubation in PBS alone (FIG. 4a) confirmed that insoluble recMoPrP aggregates had been formed following SDS treatment.

Aliquots (500 ng) of either monomeric α-helical recMoPrP or recMoPrP aggregates were slot-blotted onto Hybond-ECL nitrocellulose membrane (Amersham Biosciences) using a manifold vacuum filtration unit. The membrane was washed twice with 10 mM Tris-HCl, 150 mM NaCl, 0.05% (v/v) Tween 20 (TBST) and blocked in TBST containing 5% (w/v) non-fat dried milk powder for 60 minutes at room temperature. The membrane was then incubated in primary antibody, either mAb P1:1 or a non-PrP related mouse IgM (Sigma), at a concentration of 5 μg/ml in TBST for 60 minutes at room temperature and then washed three times in TBST (5 minutes per wash). Rabbit anti-mouse Ig HRP conjugate (DAKO), at a 1/1000 dilution in TBST, was added to the membrane, the membrane incubated at room temperature for 60 minutes, then washed three times with TBST (5 minutes per wash) and finally developed in 1-Step TMB Blotting substrate (Pierce) and incubated at room temperature for 30 minutes. From the results obtained (FIG. 4b) it was evident that mAb P1:1 bound specifically to aggregated recMoPrP with little or no binding detected to monomeric recMoPrP. The non-PrP related IgM did not bind to either aggregated or monomeric recMoPrP. These observations implied that mAb P1:1 might also be able to specifically bind PrP^{Sc} from other species thus extending the potential usefulness of mAb P1:1 both in TSE diagnosis and TSE research in general.

1. (canceled)

2. An antibody which is capable of selectively binding aggregated PrP106-126 and the abnormal disease associated PrP^{Sc} but not monomeric PrP106-126 and normal host PrP^{C}.

3. The antibody according to claim 1 wherein the antibody is polyclonal or monoclonal and of the IgG, IgM, IgD, IgE, IgA isotype or fragments thereof.

4. The antibody according to claim 1 which is capable of selectively binding to type 1 and type 2 PrP^{Sc}.

5. A hybridoma cell line, which is capable of producing a monoclonal antibody according to claim 1.

6. A method of preparing a hybridoma cell line according to claim 5 comprising the steps of:
a) providing a synthetic peptide corresponding to the amino acid sequence 106-126 of human PrP (PrP106-126) and allowing this to aggregate;
b) immunising mice with said aggregated peptide;
c) fusing splenocytes from said immunised mice to a suitable mouse myeloma partner; and
d) selecting a suitable hybridoma cell line on the basis of secreting antibody which binds to aggregated PrP106-126 but not to monomeric PrP106-126.

7. The method according to claim 6, further comprising the step of humanizing an antibody obtained from the hybridoma cell line of step d.

8. The hybridoma cell line P1:1 as deposited with the ECACC, in accordance with the Budapest Treaty, on the 6 Jun. 2006 and available under the accession number 06060601.

9. An antibody obtainable from the hybridoma cell line of claim 8.

10. (canceled)

11. A method of detecting PrP^{Sc} in a sample, comprising the steps of:
a) providing a sample of tissue extract or bodily fluid;
b) contacting said sample with an antibody according to clam 1 so that the antibody is able to bind any PrP^{Sc} present in the sample; and
c) detecting whether or not PrP^{Sc} is present in the sample by virtue of detecting antibody-PrP^{Sc} immune-complexes.

12. The method according to claim 11 wherein the antibody is labelled directly.

13. The method according to claim 11 wherein the antibody is detected indirectly either using a suitably labelled anti-mouse immunoglobulin or a suitably labelled anti-PrP antibody.

14. A kit for the detection of disease associated PrP^{Sc} in animal, especially human tissue and bodily fluids which comprises at least in part an antibody according to claim 1.

15. A method of detecting PrP^{Sc} from other species using an antibody according to claim 1.

16. (canceled)

17. (canceled)

18. An antibody according to claim 1 for use in the treatment or prophylaxis of prion diseases in humans and/or other species.

19. A method of determining the efficacy of putative prion disease therapeutic agents, comprising detecting the binding of an antibody according to claim 1 to PrP^{Sc} before and after addition of said therapeutic agent.

20. A method of treatment or prophylaxis of prion diseases in humans and/or other species, comprising providing an antibody according to claim 1 to a subject in need thereof.
